(19) United States
(12) Patent Application Publication
(10) Pub. No.: US 2021/0113747 A1
(43) Pub. Date: Apr. 22, 2021

(54) BREATHING DEVICES AND RELATED SYSTEMS AND METHODS

(71) Applicant: Science Medical, LLC, Saratoga Springs, UT (US)

(72) Inventors: John K. Bullock, Saratoga Springs, UT (US); Jeffrey L. Bullock, Saratoga Springs, UT (US)

(21) Appl. No.: 17/135,732

(22) Filed: Dec. 28, 2020

Related U.S. Application Data
(63) Continuation of application No. 15/785,248, filed on Oct. 16, 2017, now abandoned.
Application No. 15/785,248 is a continuation of application No. 14/261,250, filed on Apr. 24, 2014, now abandoned.
(60) Provisional application No. 61/815,612, filed on Apr. 24, 2013.

(51) Int. Cl. A61M 1/00; A61M 16/16; A61M 16/04; A61M 16/10; A61M 16/00

(52) U.S. Cl. CPC A61M 1/008; A61M 16/16; A61M 16/0465; A61M 16/101; A61M 16/1095; A61G 13/101; A61M 16/0051; A61M 2205/3303; A61M 2230/20; A61M 2205/3368; A61M 2205/3324; A61M 16/024

(57) ABSTRACT

Breathing devices deliver at least one treatment to a subject and include at least two components comprising at least one of a ventilation unit for supplying a gas to the subject, a humidification unit for humidifying a gas supplied to the subject, a nebulizer unit for supplying a medication to the subject, a suction unit for suctioning a portion of an airway of the subject, and a cough assist unit for simulating a cough within the subject. Methods of providing a treatment to a subject may be provided with a breathing device.

### CROSS-REFERENCE TO RELATED APPLICATION

This application claims the benefit of U.S. Provisional Patent Application Ser. No. 61/815,612, filed Apr. 24, 2013, and is a continuation of Utility patent application Ser. No. 14/261,250, filed Apr. 24, 2014.

### TECHNICAL FIELD

The present disclosure relates generally to breathing devices and related systems and methods for use in delivering at least one treatment to the subject via the trachea of the subject. In particular, the present disclosure relates generally to breathing devices including two or more components for delivering at least one treatment to the subject via the trachea of the subject and related systems and methods.

### BACKGROUND

The trachea, or windpipe, forms part of the human airway system. Airways are pipes that carry oxygen-rich air to the lungs. They also carry carbon dioxide, a waste gas, out of the lungs. When a human inhales, air travels from the nose, through the larynx, and down the windpipe. The windpipe splits into two bronchi that each enter into the lungs.

Problems with the trachea (windpipe) may include narrowing, inflammation, and some inherited conditions. A tracheotomy is a medical procedure that is designed to alleviate problems associated with the trachea. For example, a tracheotomy may be used to help a subject breathe if they have swallowing problems, or have conditions that affect coughing or block the airways. One might also need a tracheotomy if they are in critical care and need to be on a ventilator for long durations of time.

A tracheotomy is a surgical procedure to create an opening through the neck into the trachea. For long-term treatment, a tracheostomy or “trach” tube is placed through this opening thus providing an airway through the neck of the subject. The tube also provides access to the subject's lungs whereby secretions may be removed by inserting a suction tube through the trach tube.

The air breathed by tracheostomy patients does not pass through the nasal cavities, the mouth and the throat, and therefore does not receive the necessary moisture to prevent excessive drying of the trach and lungs. Further, the air is not warmed by passing through the mouth and nose. This can lead to irritation, coughing and excess mucus or “plug” for the subject by blocking the airway. Such excess mucus may also form a “plug” in the subject's airway possibly causing asphyxiation, which may lead to the subject's death. Further, some subjects with respiratory illness have weak coughs, which lead to difficulties in clearing secretions from the airway and pathogens (e.g., pneumonia or Streptococcus) cannot be cleared by coughing. Secretions can obstruct the airway making it difficult for the subject to maintain required oxygen levels.

Equipment for use in providing tracheotomy patient care varies between care facilities. The size and complexity of this equipment makes it difficult, if not impossible, for subjects to be ambulatory when they are discharged from the care facility. In many cases, subjects are thus confined to their dwellings. If care protocols are not rigidly followed, the subject may require additional treatment in the care facility.

Generally, subjects may not be discharged from a care facility until a skilled nursing center, nursing home, long-term acute care facility, or the subject's dwelling acquires the necessary equipment to provide the tracheostomy patient with the needed care. Extended stay in the primary care facility (e.g., the hospital) results in increased medical expenses. Extended stay further prevents other subjects from receiving treatment by the primary care facility.

In some instances, a discharged tracheostomy patient may experience complications that require additional medical attention and treatment in a care facility. In many instances (e.g., under the Affordable Care Act), if the subject is admitted back into the hospital within thirty days of discharge, the hospital must bear the costs associated with the additional treatment as well as the initial visit. Accordingly, there is a need to provide systems, methods and devices that reduce complications of discharged tracheostomy patients and chronic respiratory patients.

### BRIEF SUMMARY

In some embodiments, the present disclosure comprises a breathing device for delivering at least one treatment to a subject through an airway device. The breathing device includes a control unit comprising at least two components selected from the group consisting of a ventilation unit for supplying a gas to the subject, a humidification unit for humidifying a gas supplied to the subject, a nebulizer unit for supplying a medication to the subject, a suction unit for suctioning a portion of an airway of the subject, and a cough assist unit for simulating a cough within the subject. The breathing device includes at least one connecting tube in communication with the at least two components of the control unit, an airway device for accessing an airway of the subject where the airway device is coupled to the at least one connecting tube, and a control system for selectively supplying the at least one treatment to the subject with the least two components through the at least one connecting tube and the airway device.

In additional embodiments, the present disclosure comprises a portable breathing device. The breathing device includes a control unit and at least two components operatively coupled to the control unit. Each of the at least two components comprise at least one of a ventilation unit for supplying a gas to the subject, a humidification unit for humidifying a gas supplied to the subject, a nebulizer unit for supplying a medication to the subject, a suction unit for suctioning a portion of an airway of the subject, a cough assist unit for simulating a cough within the subject, an oxygen concentrator, a sensor to monitor the blood saturation level of the subject, an enteral feeding unit for supplying nutrients to the subject, and an oral care unit for use with a mouth or teeth of the subject. The breathing device includes a manifold operably coupled to an output of each of the at least two components and configured to be in communication with the airway of the subject and a control system for selectively supplying the at least one treatment to the subject with the least two components through the manifold.

In yet additional embodiments, the present disclosure comprises a method of providing a treatment to a subject with a breathing device. The method includes monitoring at least one biometric parameter associated with the subject with a control system of the breathing device where the control unit comprises at least two components for providing at least one treatment to the subject each comprising at least one of a ventilation unit for supplying a gas to the subject, a humidification unit for humidifying a gas supplied to the subject, a nebulizer unit for supplying a medication to the subject, a suction unit for suctioning a portion of an airway of the subject, and a cough assist unit for simulating a cough within the subject. In response to the monitoring at least one biometric parameter of the subject, at least one of providing an alert to at least one user regarding the at least one biometric parameter of the subject and automatically providing or ceasing at least one treatment to an airway of the subject with the control unit of the breathing device.

### BRIEF DESCRIPTION OF THE DRAWINGS

FIG. 1 shows a flowchart of a representative system that provides a suitable operating environment in which various embodiments of the present disclosure may be implemented.

FIG. 2 shows a flowchart of a representative networking system that provides a suitable environment in which various embodiments of the present disclosure may be implemented.

FIG. 3 is a schematic view of a breathing device coupled to an airway device (e.g., a tracheostomy tube) in accordance with a representative embodiment of the present disclosure.

FIG. 3A is schematic view of the housing unit 70 of the breathing device 60 shown in FIG. 3.

FIG. 4 is a perspective view of a breathing device comprising a base manifold that is configured to interchangeably receive various components or modules in accordance with a representative embodiment of the present disclosure.

FIG. 5 is a flowchart of a process for monitoring subject biometrics with a breathing device in accordance with a representative embodiment of the present disclosure.

FIG. 6 is a cross-sectional view of a tracheostomy tube having a sensor and a wireless transmitter in accordance with a representative embodiment of the present disclosure.

FIG. 7 is a flowchart of a process for wirelessly monitoring subject biometrics with a breathing device in accordance with a representative embodiment of the present disclosure.

FIGS. 8A and 8B are cross-sectional views of a one-way valve coupled to a tracheostomy tube and a corrugated tube of a breathing device in accordance with a representative embodiment of the present disclosure.

FIG. 9 is a schematic view of a breathing device coupled to a tracheostomy tube in accordance with a representative embodiment of the present disclosure.

FIG. 10 is a cross-sectional view of a portion of the breathing device shown in FIG. 9.

### DETAILED DESCRIPTION

The presently preferred embodiments of the described disclosure will be best understood by reference to the figures, wherein like reference numbers indicate identical or functionally similar elements. It will be readily understood that the components of the present disclosure, as generally described and illustrated in the figures, could be arranged and designed in a wide variety of different configurations. Thus, the following more detailed description is not intended to limit the scope of the disclosure as claimed, but is merely representative of some embodiments of the disclosure.

The illustrations presented herein are not actual views of any particular stimulation device or component thereof, but are merely idealized, schematic representations that are employed to describe embodiments of the present disclosure.

The present disclosure relates generally to systems, devices, and methods to assist in the recovery and treatment (e.g., long-term treatment) of subjects (e.g., patients) by delivering at least one treatment to the subject via an airway (e.g., the trachea, within the nose and/or the mouth, the pharynx, the larynx, etc.) of the subject. For example, breathing devices and related systems and methods as disclosed herein may be utilized with subjects that have an airway formed through a stoma (e.g., tracheostomy) extending from an anterior portion of the neck to the trachea. It is noted while the exemplary embodiments discussed below generally reference an airway device such as tracheostomy tube for the purposes of illustration, in other embodiments, breathing devices and related systems and methods as disclosed herein may be utilized with other airway devices in communication with a subject's airway, such as, for example, a mask, an inhalation tent, an air hood, a nasal cannula, a tracheal tube, or other type of breathing tube in place of the tracheostomy tube. Such subjects may have undergone one or more of tracheotomy and laryngectomy medical procedures and/or may have chronic respiratory illnesses or diseases or other underlying medical conditions, such as, for example, acute bronchitis, acute respiratory distress syndrome (ARDS), amyotrophic lateral sclerosis (ALS), asbestosis, asthma, bronchiectasis, bronchiolitis, bronchiolitis obliterans organizing pneumonia (BOOP), bronchopulmonary dysplasia, byssinosis, chronic bronchitis, coccidioidomycosis (COCCI), chronic obstructive pulmonary disease (COPD), cryptogenic organizing pneumonia (COP), cystic fibrosis, emphysema, hantavirus pulmonary syndrome, histoplasmosis, human metapneumovirus, hypersensitivity pneumonitis, influenza, lung cancer, lymphangiomatosis, mesothelioma, nontuberculosis Mycobacterium, pertussis, pneumoconiosis (black lung disease), pneumonia, primary ciliary dyskinesia, primary pulmonary, hypertension, pulmonary arterial hypertension, pulmonary fibrosis, pulmonary vascular disease, respiratory syncytial virus, sarcoidosis, severe acute respiratory syndrome, silicosis, sleep apnea, sudden infant death syndrome, tuberculosis, pneumothorax, hypoxaemia, sinusitis, rhinosinusitis, allergic rhinitis, muscular dystrophy, spinal cord injury, other physical difficulties or injuries, or combinations thereof.

In some embodiments, the present disclosure includes an integrated, portable, modular, mobile, ambulatory, lightweight, compartmentalized, and compact medical device that provides one or more suction, humidification, nebulization, oxygen, ventilation, cough assistance, enteral feeding pump, oral care, biometric sensing, and computer software for detecting, monitoring, and controlling the breathing device to meet the needs of the subject as well as provide real-time biometric data.

In some implementations of the present disclosure, a breathing device is provided having an airway device (e.g., a tracheostomy tube) that includes a distal end for insertion into the trach opening or stoma (e.g., tracheostomy) of the subject, and a proximal end that is coupled to a base or housing unit. In other embodiments, the breathing device may be connected to the airway of the subject via the subject's nose and/or mouth. The breathing device may include one or more sensors that are configured to monitor various parameters of the subject, such as oxygen blood saturation levels, air pressure, air temperature, air humidity, volume of air displaced, rate of flow, pH level, and audio monitoring of noises originating from the subject. In some embodiments, the one or more sensors are positioned in the tracheostomy tube and in communication with air that is moving through the tube. In other embodiments, the one or more sensors are positioned on a terminal end of the tracheostomy tube, such that the one or more sensors are within the airway of the subject. Some implementations of the present disclosure include a computer device that is configured to receive (e.g., monitor) a signal from the one or more sensors and control (e.g., adjust) the breathing device as needed to meet the needs and comfort of the subject. In other embodiments, the breathing device of the present disclosure comprises manual controls for adjusting the breathing device as needed to meet the needs and comfort of the subject. In some embodiments, the breathing device comprises digital controls that may be adjusted via a touchscreen or remotely using a smart device, such as a cell phone, tablet computer, smart watch, or other computer. The breathing device may record data regarding the subject (e.g., breathing cycles, patterns, and/or status of the subject) and the use of the various features of the breathing machine (e.g., to create a usage log for tracking the subject's compliance or noncompliance with a prescribed or recommended treatment schedule).

In some embodiments, the housing unit may include a positive pressure pump (e.g., a ventilator for mechanically assisting or replacing spontaneous breathing). The ventilator is configured to provide air to the subject and maintain positive pressure in the airway device (e.g., tracheostomy tube and/or mouth hose) during the subject's exhale. This feature may reduce the occurrence of (e.g., prevent) the subject from rebreathing exhaled gases (e.g., CO₂ gases) and contamination of the breathing device that may occur from the exhaled gases. In some embodiments, the present disclosure includes a one-way valve that permits passage of air from the positive pressure pump into the subject's airway, and vents exhaled gases into the environment. In some embodiments, the ventilator may be a passive system where the ventilator provides a set amount of airflow to the subject. In other embodiments, the ventilator may include an active system where the ventilator monitors one or more parameters from the subject (e.g., monitors the parameters regarding the subject's exhale) and adjusts the amount and/or composition of the airflow accordingly.

The device includes various components (e.g., modular components) that may be selectively utilized with the airway device (e.g., tracheostomy tube) and, if implemented, the positive pressure pump, to provide the subject with comfort and medical care. For example, the breathing device comprises a humidifier component that adds humidity to the air being provided to the subject (e.g., via the positive pressure pump). In some embodiments, the humidifier component may include a heating element for increasing the temperature of the air as it is humidified. In some embodiments, the humidifier nebulizes and condenses the moisture in the air from the positive pressure pump prior to the air being administered to the subject (e.g., via the tracheostomy tube, via a tube or mask in communication with the subject's nose or mouth). In such an embodiment, the humidifier component is configured to produce a “cloud” of humid air (e.g., water vapor) that is inhaled by the subject. The humid air thins pulmonary secretions and reduces irritation due to dryness and may reduce the occurrence of plugs in the subject's airway. In some embodiments, the humidifier component is configured to treat the air with sufficient moisture to achieve an optimal level of humidity for the subject. For example, the humidifier is configured to maintain a level of humidity for the air at 44 mg/L or higher. The breathing device may sense, receive, and record data regarding the humidity for the air (e.g., inside the trachea and/or nasal/mouth airways of the subject) and may automatically adjust the output of the humidifier component based on the collected data.

The humidifier component may adjust the temperature of the moisture that is delivered to the subject. For example, the humidifier component comprises a heating coil that warms the humidified air to a desired temperature prior to administration. In other embodiments, a tube (e.g., the tracheostomy tube) of the breathing device may include a heating element or wire that is embedded within the wall of the tube, and is configured to heat the tube when a current is applied to the heating element. In some embodiments, the humidifier maintains the temperature of the moisture and air at 37° C. In some embodiments, the heating element may be able to heat the air in the range between 37° C. and 100° C.

Some implementations of the present disclosure include a nebulization component that may be attached to or incorporated within the breathing device to administer a medication to the subject via the tracheostomy tube or mask. In some embodiments, using one or more sensors (e.g., that sense airflow at a tracheostomy tube or mask), the breathing apparatus may monitor breathing of the subject determine when medication is to be administered to the subject (e.g., by sounding an alarm or by automatically delivering the medication).

Some implementations of the present disclosure include an oxygen component that may be attached to the breathing device to administer oxygen to the subject. In some embodiments, the oxygen component utilizes an oxygen concentrator that pulls oxygen from the atmosphere. If an oxygen concentrator is incapable of supplying the subject with sufficient oxygen, the breathing device may be coupled to an oxygen canister. In some embodiments, the breathing device may use a sensor to monitor the blood saturation level of the subject (e.g., a pulse oximeter) and may adjust the rate of flow of the oxygen as well as the concentration levels of the provided oxygen to be adjusted manually, electronically, or through a control system of the breathing device. If the blood saturation level is not optimal, the breathing device may sound an alarm and/or send notifications regarding the detected level.

Some implementations of the present disclosure include a negative pressure pump that is configured to remove secretions from the subject's airway. The negative pressure pump is coupled to a suction cannula that is inserted through the tracheostomy tube and provides a negative pressure to the subject's airway. In some embodiments, the breathing device may include a yankauer to suction the subject's mouth. The negative pressure pump includes a vacuum gauge and may include various suction settings (e.g., adjustable between 50 and 500 mm Hg). For example, the negative pressure pump comprises an adult suction setting of 80-120 mm Hg, a child suction setting of 80-100 mm Hg, and an infant suction setting of 60-80 mm Hg

In some embodiments, the breathing device may oscillate between ventilation and suction automatically or manually if a ventilator is needed to increase the level of pressure in the subject's lungs. The breathing device may alert the subject as to predetermined suction times and durations (e.g., by monitoring the subject's breathing). In some embodiments, the breathing device may utilize a pressure sensor that monitors that pressure in the subject's airway. For example, the breathing device may sense if a pressure inside the airway is elevated above the predetermined level and may notify the subject and/or caregiver.

In some embodiments, the breathing device provides automated suctioning based upon pressure readings detected during the subject's breathing pattern or is a selected audio signal is detected from the subject (e.g., via a microphone sensor of the breathing device, a microphone and/or speakers of a device separate from the breathing device, e.g., a smartphone) indicating the subject requires suctioning. For example, the tracheostomy tube comprises a pressure sensor that detects changes in air pressure due to excessive secretions in the subject's airway. When a change in air pressure is detected, the breathing device automatically advances the suction cannula through the tracheostomy tube and into the subject's airway to apply negative pressure and remove the excess pulmonary secretions. In some embodiments, the positive pressure pump and oxygen component restore oxygen to the subject between suctioning cycles (e.g., by insufflating the lungs).

In some embodiments, the breathing device may include a cough assist feature. For example, the breathing device may apply a positive pressure to the subject's air followed by a negative pressure. Such a feature may emulate a cough by assisting the subject in at least partially clearing secretions within the subject's airway.

In some embodiments, the breathing device may include a feature for use with a feeding device for the subject. For example, the breathing device may include a pump for delivering nutrition to a subject (e.g., an enteral feeding pump).

In some embodiments, the breathing device may include one or more oral care devices for a subject.

In some embodiments, the breathing device may include one or more devices (e.g., one or more pumps) that are utilized for more than one of the various components of the breathing device. For example, the breathing device may include one or more common pumps that facilitate operation of one or more of the ventilation unit, humidification unit, nebulizer unit, suction unit, cough assist unit, an oxygen concentrator unit, a pulse oximeter unit, sensor to monitor the blood saturation level of the subject enteral feeding unit, and oral care unit.

In some embodiments, the present disclosure is configured to provide an alert when the subject's biometric levels vary from a desired or prescribed level. For example, the breathing device provides an audible alarm when the subject's blood oxygen falls below a desired level. In other embodiments, the breathing device sends a communication to a portable computer device (e.g., smart phones, tablets, pads, computers, smart watches, and other electronic devices) when an undesirable signal or level is detected. In some embodiments, the breathing device sends an alert or message to a caregiver (e.g., a nurse's station), or otherwise alerts the caregiver when an undesirable signal or level is detected. The caregiver may then adjust the breathing device to correct the undesirable levels. For example, the caregiver may adjust the breathing device directly with a control system interface provided on the breathing device or remotely via a software application on a computer device, such as a personal computer, a tablet computer, a smart phone, a smart watch, a mobile phone, or other device.

Some embodiments of the present disclosure are configured to detect a plug or occlusion of the subject's airway and/or the tracheostomy tube. For example, a pressure sensor may monitor and detect a change in air pressure, which indicates that the subject is no longer breathing, or having difficulty breathing due to an occlusion. The breathing device may be configured to generate an alert, whereby a care provider is alerted to the plugged status of the subject's airway or tracheostomy tube. The care provider may then take action to clear the plug and restore normal breathing to the subject. It will be appreciated that an occlusion or blockage may be detected by monitoring various other biometric parameters in addition to, or in place of, air pressure.

In some embodiments, the control system of the breathing device may be configured to sound an alarm when one or more components of the device (e.g., the components, tubes, or units discussed below, which may be disposable) are in need of replacing.

As mentioned above, some embodiments of the present disclosure are configured for use with a computer device, whereby the computer device enables a user to receive, interpret, adjust, and monitor the settings of the breathing device to maintain the comfort and care of the subject.

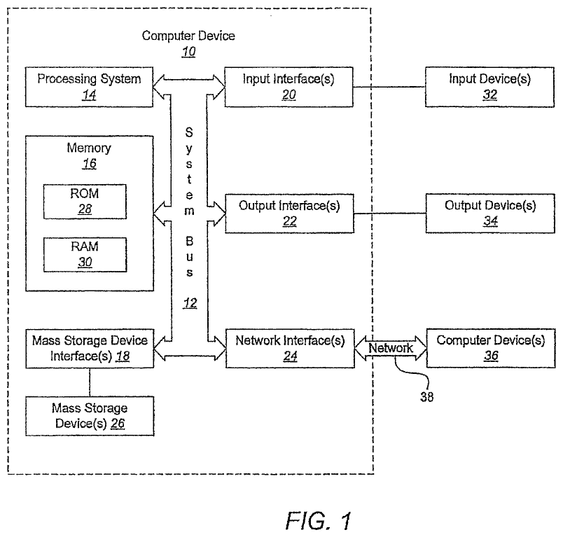

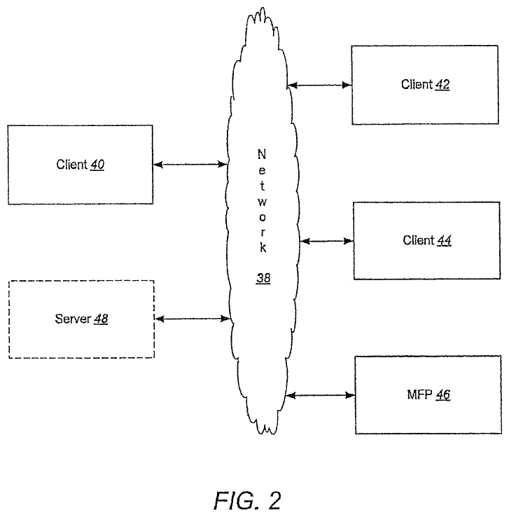

Referring now to FIGS. 1 and 2 and the corresponding discussion, which is intended to provide a general description of a suitable operating environment in which embodiments of the disclosure may be implemented. One skilled in the art will appreciate that embodiments of the disclosure may be practiced by one or more computing devices and in a variety of system configurations, including in a networked configuration. However, while the methods and processes of the present disclosure have proven to be particularly useful in association with a system comprising a general purpose computer, embodiments of the present disclosure include utilization of the methods and processes in a variety of environments, including embedded systems with general purpose processing units, digital/media signal processors (DSP/MSP), application specific integrated circuits (ASIC), standalone electronic devices, and other such electronic environments.

Embodiments of the present disclosure may include one or more computer readable media, wherein each medium may be configured to include or includes thereon data or computer executable instructions for manipulating data. The computer executable instructions include data structures, objects, programs, routines, or other program modules that may be accessed by a processing system, such as one associated with a general-purpose computer capable of performing various different functions or one associated with a special-purpose computer capable of performing a limited number of functions. Computer executable instructions cause the processing system to perform a particular function or group of functions and are examples of program code means for implementing acts for methods disclosed herein. Furthermore, a particular sequence of the executable instructions provides an example of corresponding acts that may be used to implement such acts. Examples of computer readable media include random-access memory (“RAM”), read-only memory (“ROM”), programmable read-only memory (“PROM”), erasable programmable read-only memory (“EPROM”), electrically erasable programmable read-only memory (“EEPROM”), compact disc read-only memory (“CD-ROM”), or any other device or component that is capable of providing data or executable instructions that may be accessed by a processing system.

With reference to FIG. 1, a representative system for implementing embodiments of the disclosure includes computer device 10, which may be a general-purpose or special-purpose computer. For example, computer device 10 may be a personal computer, a notebook computer, a personal digital assistant (“PDA”) or other hand-held device, a tablet, a workstation, a minicomputer, a mainframe, a supercomputer, a multi-processor system, a network computer, a processor-based consumer electronic device, a smart phone, a smart watch, or the like.

Computer device 10 may include a system bus 12, which may be configured to connect various components thereof and enables data to be exchanged between two or more components. System bus 12 may include one of a variety of bus structures including a memory bus or memory controller, a peripheral bus, or a local bus that uses any of a variety of bus architectures. Typical components connected by system bus 12 include processing system 14 and memory 16. Other components may include one or more mass storage device interfaces 18, input interfaces 20, output interfaces 22, and/or network interfaces 24, each of which will be discussed below.

Processing system 14 includes one or more processors, such as a central processor and optionally one or more other processors designed to perform a particular function or task. It is typically processing system 14 that executes the instructions provided on computer readable media, such as on memory 16, a magnetic hard disk, a removable magnetic disk, a magnetic cassette, an optical disk, thumb drives, solid state memory, a universal serial bus or from a communication connection, which may also be viewed as a computer readable medium.

Memory 16 includes one or more computer readable media that may be configured to include or includes thereon data or instructions for manipulating data, and may be accessed by processing system 14 through system bus 12. Memory 16 may include, for example, ROM 28, used to permanently store information, and/or RAM 30, used to temporarily store information. ROM 28 may include a basic input/output system (“BIOS”) having one or more routines that are used to establish communication, such as during start-up of computer device 10. RAM 30 may include one or more program modules, such as one or more operating systems, application programs, and/or program data.

One or more mass storage device interfaces 18 may be used to connect one or more mass storage devices 26 to system bus 12. The mass storage devices 26 may be incorporated into or may be peripheral to computer device 10 and allow computer device 10 to retain large amounts of data. Optionally, one or more of the mass storage devices 26 may be removable from computer device 10. Examples of mass storage devices include hard disk drives, magnetic disk drives, thumb drive tape drives and optical disk drives. A mass storage device 26 may read from and/or write to a magnetic hard disk, a removable magnetic disk, a magnetic cassette, an optical disk, or another computer readable medium. Mass storage devices 26 and their corresponding computer readable media provide nonvolatile storage of data and/or executable instructions that may include one or more program modules such as an operating system, one or more application programs, other program modules, or program data. Such executable instructions are examples of program code means for implementing acts for methods disclosed herein.

One or more input interfaces 20 may be employed to enable a user to enter data and/or instructions to computer device 10 through one or more corresponding input devices 32. Examples of such input devices include a keyboard and alternative input devices, such as a mouse, trackball, light pen, stylus, or other pointing device, a microphone, a joystick, a game pad, a touchscreen, a keypad, a satellite dish, an RFID chip, a scanner, a camcorder, a digital camera, and the like. Similarly, examples of input interfaces 20 that may be used to connect the input devices 32 to the system bus 12 include a serial port, a parallel port, a game port, a universal serial bus (“USB”), an integrated circuit, a firewire (IEEE 1394), or another interface. For example, input interface 20 includes an application specific integrated circuit (ASIC) that is designed for a particular application. In a further embodiment, the ASIC is embedded and connects existing circuit building blocks.

One or more output interfaces 22 may be employed to connect one or more corresponding output devices 34 to system bus 12. Examples of output devices include a monitor or display screen, a speaker, a printer, a multi-functional peripheral, and the like. A particular output device 34 may be integrated with or peripheral to computer device 10. Examples of output interfaces include a video adapter, an audio adapter, a parallel port, and the like.

One or more network interfaces 24 enable computer device 10 to exchange information with one or more other local or remote computer devices, illustrated as computer devices 36, via a network 38 that may include hardwired and/or wireless links. Examples of network interfaces include a network adapter for connection to a local area network (“LAN”) or a modem, wireless link, or other adapter for connection to a wide area network (“WAN”), such as the Internet. The network interface 24 may be incorporated with or peripheral to computer device 10. In a networked system, accessible program modules or portions thereof may be stored in a remote memory storage device. Furthermore, in a networked system computer device 10 may participate in a distributed computing environment, where functions or tasks are performed by a plurality of networked computer devices.

Thus, while those skilled in the art will appreciate that embodiments of the present disclosure may be practiced in a variety of different environments with many types of system configurations, FIG. 2 provides a representative networked system configuration that may be used in association with embodiments of the present disclosure. The representative system of FIG. 2 includes a computer device, illustrated as client 40, which is connected to one or more other computer devices (illustrated as client 42 and client 44) and one or more peripheral devices (illustrated as multifunctional peripheral (MFP) 46) across network 38. While FIG. 2 illustrates an embodiment that includes a client 40, two additional clients, client 42 and client 44, one peripheral device, MFP 46, and optionally a server 48, connected to network 38, alternative embodiments include more or fewer clients, more than one peripheral device, no peripheral devices, no server 48, and/or more than one server 48 connected to network 38. Other embodiments of the present disclosure include local, networked, or peer-to-peer environments where one or more computer devices may be connected to one or more local or remote peripheral devices. Moreover, embodiments in accordance with the present disclosure may include a single electronic consumer device, wireless networked environments, and/or wide area networked environments, such as the Internet, a cellular network, and/or a messaging service (e.g., a geographic messaging service (GMS).

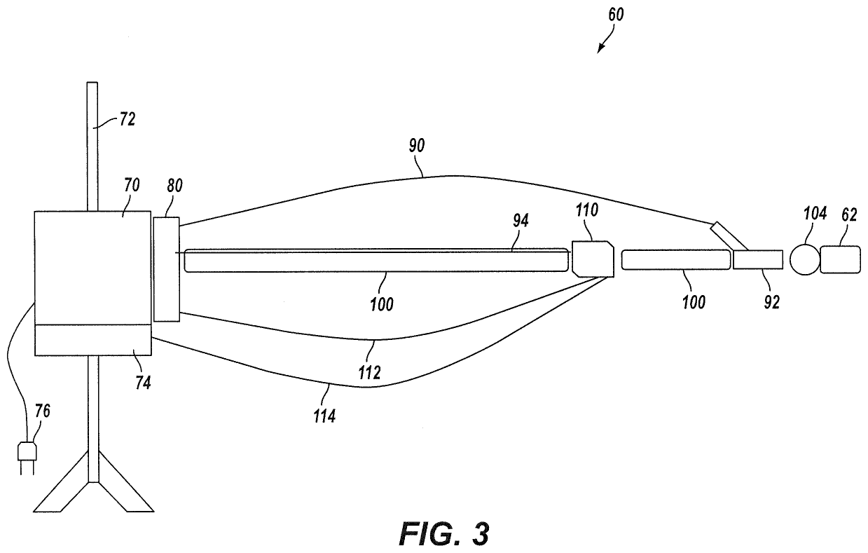

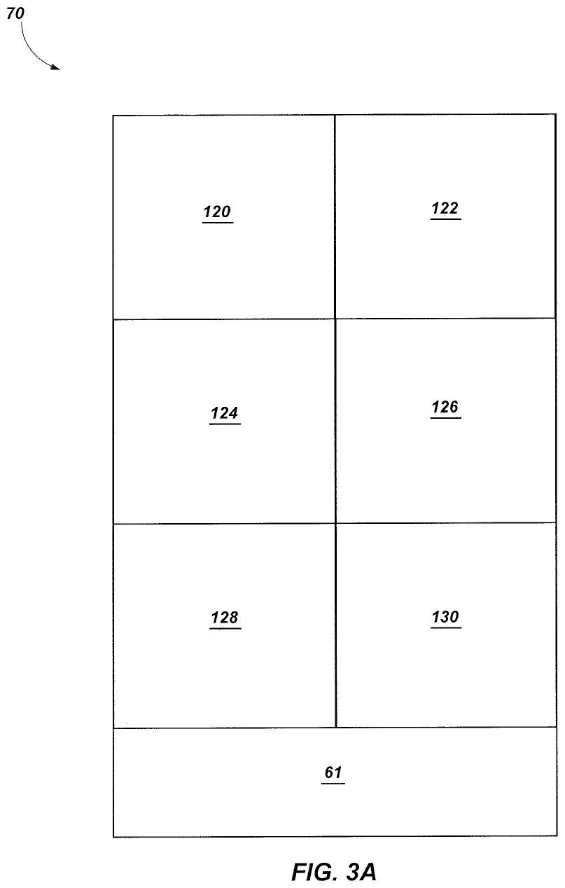

FIG. 3 is a schematic view of a breathing device 60 coupled to a tracheostomy tube 62 and FIG. 3A is schematic view of the housing unit 70 of the breathing device 60. Referring to FIGS. 3 and 3A, a breathing device 60 is shown. In some embodiments, breathing device 60 comprises a control or housing unit 70 that may be configured to attach to an IV pole 72. In other embodiments, housing unit 70 comprises a freestanding unit that may be placed on a flat surface, such as a table, a shelf, or floor surface. Further, in some embodiments, housing unit 70 comprises wheels or casters to assist in moving and positioning the device 60. Further still, in some embodiments, housing unit 70 comprises a carrying case to assist the user in transporting the device 60.

In some embodiments, housing unit 70 comprises an outer shell having an interior configured to store various modular components. In some embodiments, housing unit 70 comprises a sealed unit in which is permanently stored a variety of components. For example, the user may not remove or add components to housing unit 70. In other embodiments, housing unit 70 may be accessed by a user to selectively insert and remove various components dependent upon the needs of the user and/or subject. Thus, in some embodiments, housing unit 70 may be configured by a user to include one or more modular components, thereby providing a breathing unit 60 that is customized according to the user's specifications.

Housing unit 70 may comprise any material that is compatible for use in a medical or clinical setting. For example, housing unit 70 comprises a plastic or metal material that is shaped and sized for convenient and portable use. Housing unit 70 may comprise a single, monolithic structure, or may comprise multiple sections that are joined together to provide a final structure.

In some embodiments, housing unit 70 comprises a power converter whereby housing unit 70 may be plugged (e.g., with wall plug 76) into an electrical receptacle to power the unit 70. In other embodiments, housing unit 70 comprises a battery 74 that is configured to power the various components of breathing device 60. In some embodiments, battery 74 is charged by plugging wall plug 76 into an electrical receptacle. In some embodiments, the housing unit 70 may be configured to be plugged into a vehicle battery for mobile use and/or mobile charging of the battery 74.

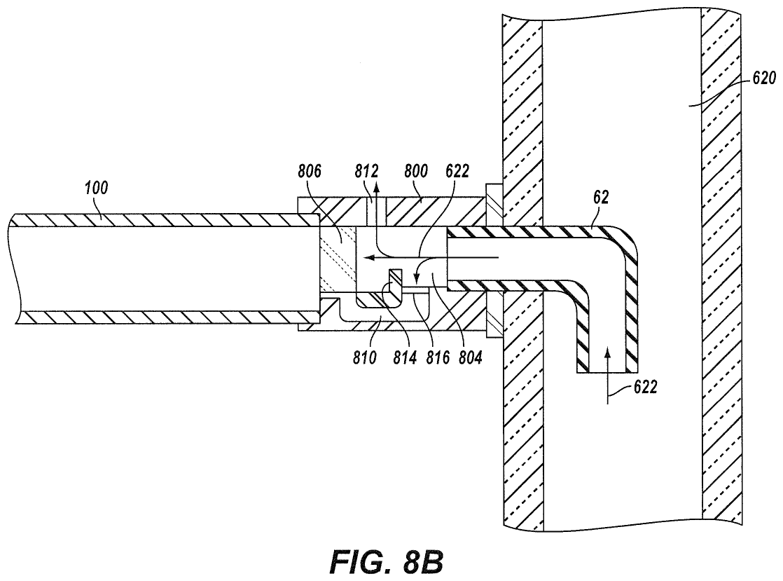

The breathing device 60 may include a manifold 80. Manifold 80 provides an interface between the modular components stored within housing unit 70, and the various parts of breathing device 60 that are external to housing unit 70. Manifold 80 may comprise any structure or format that provides access to the modular components. In some embodiments, manifold 80 comprises a portion of the outer shell of housing unit 70. For example, the manifold 80 may be separated from the housing unit 70 such the manifold 80 may move (e.g., rotate and/or translate) relative to the housing unit 70. Such an embodiment may enable the tubing and/or wiring, which may be suspended on a swivel arm (see, e.g., FIG. 9) between the manifold 80 and the housing unit 70 to comply with movement of the subject in order to reduce the occurrence of tangling the tubing and/or wiring of the breathing device 80. In other embodiments, manifold 80 is coupled to the outer surface of housing unit 70. Further, in some embodiments, manifold 80 is operably connected to housing unit 70 and the various modular components via a system of electrical cords and tubing (see, e.g., FIG. 9).

The manifold 80 comprises a plurality of sockets, receptacles, and/or couplers that are configured to receive or support inputs and output of the various components of the breathing device 60 (e.g., tubing and electrical cords for components and parts of breathing device 60). In some embodiments, the sockets, receptacles and/or couplers of manifold 80 are color coded to the various modular components and related accessories to facilitate the user in making proper connections when setting up breathing device 60. For example, breathing device 60 comprises a suction tube 90 having a distal end that is coupled to the subject's tracheostomy tube 62, and having a proximal end that is coupled to manifold 80. The socket into which the proximal end of suction tube 90 is inserted into manifold 80 is directly coupled to a modular component comprising suction unit (e.g., a vacuum or suction pump 124) that is stored within housing unit 70. Accordingly, by coupling suction tube 90 to manifold 80, pulmonary secretions are removed from the subject's airway and delivered to housing unit 70 via suction tube 90 and manifold 80. Manifold 80 comprises sockets and/or other coupling means for receiving the remaining components and parts, as is discussed below.

In some embodiments, the breathing device 60 may control the manifold 80 such that the control system of the breathing device 60 may open and close various portions of the manifold 80 (e.g., a portion of the tubing of one or more components of the breathing device 60 extending through a valve of the manifold 80) to facilitate operation of particular components of the breathing device 60. For example, when the ventilation unit (e.g., positive pressure pump 120) of the breathing device 60 is to be used, the breathing device 60 may act to open a respective valve in the manifold 80 to enable operation of the ventilation unit (e.g., to enable positive pressure pump 120 to communicate with corrugated tubing 100).

With continued reference to FIGS. 3 and 3A, in some embodiments, breathing device 60 comprises a section of tubing (e.g., corrugated tubing 100) having a proximal end coupled to manifold 80 and a distal end that is coupled to the subject's trach tube 62. Tubing 100 may comprise any material that is compatible for use in a medical or clinical setting. In some embodiments, tubing 100 comprises a flexible polymer material having a metal or hard plastic coiling embedded within a sidewall of the tubing. Further, in some embodiments, tubing 100 comprises a single lumen through which air and humidity are delivered to the subject from housing unit 70. In other embodiments, tubing 100 comprises a first lumen configured to deliver air and humidity to the subject, and a second lumen configured to remove exhaled gases from the subject.

In some aspects of the disclosure, housing unit 70 comprises a positive pressure pump 120 that pushes a fluid (e.g., a gas, such as air, e.g., atmospheric air, oxygen, or combinations thereof) through tubing 100 to provide positive air pressure at tracheostomy tube 62. Positive air pressure may be desirable to prevent expired air from being rebreathed by the subject. Positive pressure may further prevent pathogens and pulmonary secretions from entering tubing 100 via tracheostomy tube 62. Further still, positive pressure may be useful for advancing air, humidity, and nebulized medications through tube 100 and into the subject's airway.

In some embodiments, the positive pressure pump 120 of the housing unit 70 provides positive pressure within tubing 100 from approximately 2 liters/minute (L/min) to approximately 12 L/min. In other embodiments, housing unit 70 provides positive pressure within tubing 100 from approximately 4 L/min to approximately 8 L/min. In some embodiments, housing unit 70 provides positive pressure within tubing 100 of approximately 6 L/min.

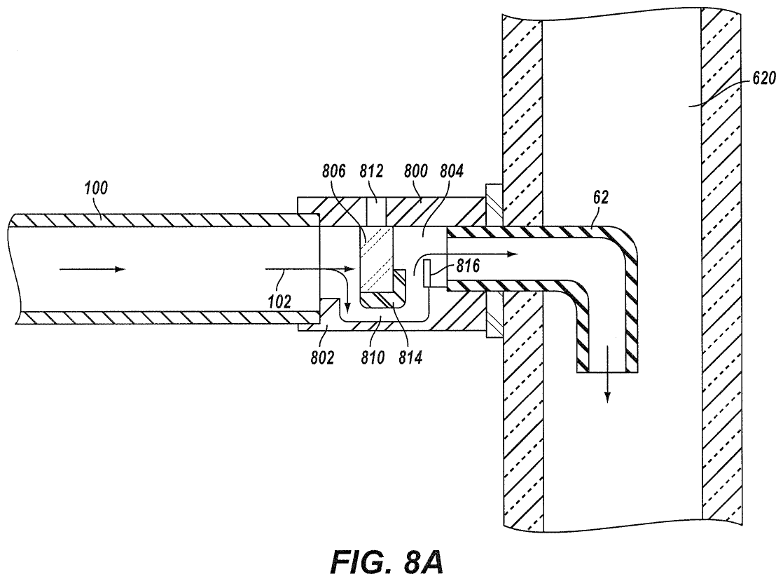

In some embodiments, breathing device 60 comprises a one-way valve 104 that is positioned between tracheostomy tube 62 and tubing 100. One-way valve 104 permits passage of fresh air from tubing 100 during inhalation, and prevents passage of expired air into tubing 100. In some embodiments, one-way valve 104 releases expired (e.g., exhaled) air into the environment. In other embodiments, one-way valve 104 directs expired air into a second lumen of tubing 100 that is configured to remove exhaled gases from the subject and breathing device 60. A non-limiting example of a one-way valve is shown and discussed below in connection with FIGS. 8A and 8B.

Breathing device 60 may comprise an inline unit 110 that may act as at least one of a nebulizer and a humidifier. In other embodiments, breathing device 60 may comprise a modular unit 122 that may act as at least one of a nebulizer and a humidifier that is stored within housing unit 70. Unit 110, 122 is configured to provide a cloud of water vapor to the subject via tubing 100. In some embodiments, inline unit 110 is positioned on tubing 100 such that a minimum distance (e.g., 6 to 10 inches (15.24 to 25.4 centimeters)) is provided between unit 110 and the subject. In such an embodiment, this distance may provide sufficient charging of the air prior to inhalation by the subject. For example, this distance permits sufficient charging of the air with water vapor. In other embodiments, this distance permits sufficient charging of the air with a medication. In some embodiments, unit 110 comprises a secondary positive pressure pump configured to push the vapor cloud through tubing 100 and into the subject's airway.

Unit 110, 122 may comprise any type, style or model of nebulizer and/or humidifier device. In some embodiments, unit 110, 122 comprises a vibrating mesh nebulizer, a jet nebulizer, or an ultrasonic wave nebulizer. In some embodiments, unit 110, 122 may comprise a dedicated humidifier unit. In other embodiments, unit 110, 122 comprises a nebulizer and humidifier hybrid device.

In some embodiments, nebulizer/humidifier unit 110 is coupled to tubing 100 at a position between the subject and manifold 80. Water is delivered to unit 110 via water line 112 having a proximal end coupled to manifold 80 and a distal end operably attached to unit 110. Electricity is also supplied to unit 110 via a power cord 114 that is coupled to housing unit 70 or battery 74. In some embodiments, water line 112 and power cord 114 are separated from tubing 100, as shown. In other embodiments, water line 112 and power cord 114 are coupled to the outer surface of tubing 100. Further, in some embodiments, water line 112 and power cord 114 are molded into the sidewall of tubing 100, wherein the manifold socket for tubing 100 comprises electrical contacts for power cord 114, and water supply for water line 112.

As previously mentioned, in some embodiments, breathing device 60 comprises a suction tube 90 and suction pump 124 for removing pulmonary secretions from the subject's airway. In some embodiments, suction tube 90 is coupled to tubing 100 at a position between manifold 80 and tracheostomy tube 62. In some embodiments, tubing 100 comprises a suction adapter 92 having a Y-port through which suction tube 90 is inserted into tubing 100 and the airway of the subject via tracheostomy tube 62.

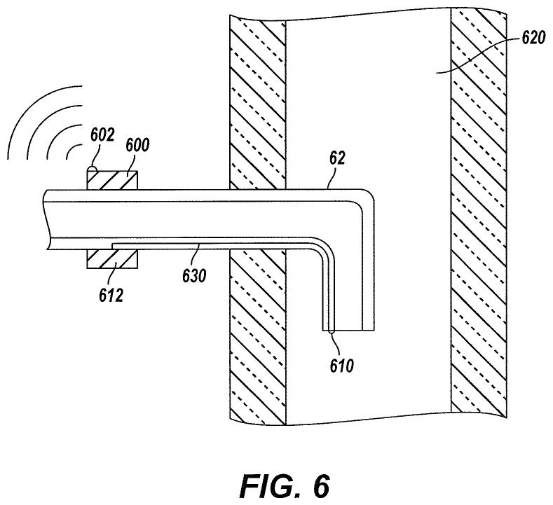

Suction tube 90 may be manually advanced through suction adapter 92 and tracheostomy tube 62, and into the subject's airway. In some embodiments, suction tube 90 is automatically advanced into the subject's airway when breathing device 60 detects a change in air pressure in the subject's airway. For example, breathing device 60 may comprise air pressure sensors (e.g., sensors 610 (FIG. 6)) that monitor the air pressure of the subject's airway. When a reduction in air pressure is detected by the air pressure sensor, breathing device 60 automatically advances suction tube 90 into the subject's airway to remove pulmonary secretions. Following removal of the secretions, the positive pressure pump 120 provides the subject with air and oxygen to restore proper levels. In some embodiments, a suctioning event comprises multiple alternating stages of suctioning and positive air pressure (e.g., via positive pressure pump 120) to clear pulmonary secretions from the subject's airway and ensure proper air and oxygen levels for the subject.

In some embodiments, the breathing device 60 may include a sensor (e.g., sensors 610 (FIG. 6)) that may monitor the pH level in condensates produced by the subject (e.g., condensates received form the subject during a suctioning operation or a cough assist. Such detection of the pH level of condensates and/or exhaled gases of the subject may be utilized to determine if the subject has an indication of an infection in the subject's airway.

In some embodiments, the breathing device 60 may include a sensor (e.g., sensors 610 (FIG. 6)) in a component of the breathing device 60 (e.g., the tracheostomy tube 62) that may act as a microphone to monitor noises originating from the subject. Such detection of noises originating from the subject may be utilized to determine, for example, if the subject is having difficulty breathing or is developing a plug in the subject's airway. In some embodiments, the breathing device 60 may relay the audio from the sensor to a speaker on the breathing device 60 or to a remote device separate from the breathing device 60.

In some embodiments, breathing device 60 comprises means for heating the air in tubing 100. For example, tubing 100 comprises a heating element 94 that is embedded within the sidewall of tubing 100. The heating element 94 comprises a proximal end that is attached to manifold 80 and receives and electrical current from either battery 74 or housing unit 70. The electrical current causes the temperature of the heating element 94 to increase thereby warming the portion of tubing 100 in which the heating element 94 is embedded. As the temperature of tubing 100 increases, the air within tubing 100 is warmed to a desired temperature. In some embodiments, the heating element 94 warms the air and/or water vapor to approximately 37° C.

In some embodiments, tubing 100, humidifier 110, tracheostomy tube 62, and/or another portion of breathing device 60 comprises a temperature sensor (e.g., sensors 610 (FIG. 6)) that detects the temperature of air and/or humidity moving through tubing 100. Breathing device 60 may include a thermostat that is coupled to the heating element 94 and the temperature sensor, whereby a user may set the thermostat to a desired temperature, and whereby the thermostat monitors the air temperature and automatically adjusts the electrical current running through the heating element 94 to achieve and maintain the desired temperature.

In some embodiments, tubing 100 comprises an inner lumen and an outer lumen, as previously mentioned. As expired gases move through the inner lumen, heat from the expired gases is transferred to the air within the outer lumen, thereby warming the air and humidity to an optimal temperature for the subject. In some embodiments, expired gases move through the outer lumen while fresh air and oxygen is delivered to the subject via the inner lumen. Further, in some embodiments, tubing 100 comprises two or more tubes, wherein each tube is configured to accommodate air and/or liquid in an isolated manner.

In some embodiments, the breathing device 60 may include a cough assist unit 126. For example, the breathing device 60 may apply a positive pressure to the subject's airway followed by a negative pressure. Such a feature may emulate a cough by assisting the subject in at least partially clearing secretions within the subject's airway. In some embodiments, such a negative and positive pressure may be produced with a pump of the cough assist unit 126. In other embodiments, one or more other components of the breathing device 60 may be utilized to produce such a negative and positive pressure. For example, such a negative and positive pressure may be produced with the pump or pumps of one of more of the positive pressure pump 120, the nebulizer and/or humidifier unit 110, 122, the suction pump 124, or combinations thereof.

In some embodiments, the breathing device 60 may include a feature for use with a feeding device for the subject. For example, the breathing device 60 may include a feeding unit 128 for delivering nutrition to a subject (e.g., an enteral feeding pump).

In some embodiments, the breathing device may include oral care unit 130 including one or more oral care devices for a subject (e.g., dental scaling, cleaner, and/or polishers, dental water jets, suction devices, other oral or tongue cleaners, etc.).

Breathing device 60 includes a control system 61 (e.g., computer device 10 (FIG. 1)) including an input device (e.g., input device 32 (FIG. 1)) and an output device (e.g., output devices 34 (FIG. 1)) that may be utilized to control one or more components of the breathing device 60.

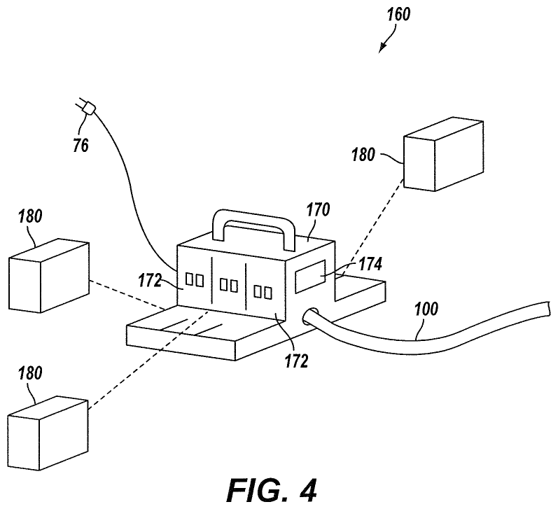

Referring now to FIG. 4, a portable breathing device 160 is shown. In some embodiments, the portable breathing device 160 may be similar to and include similar components and functions of the breathing device 60 discussed above in relation to FIGS. 3 and 3A. The breathing device 160 comprises a portable housing 170 having a plurality of docking stations 172 configured to operably receive one or more modular components 180. Docking stations 172 are configured to provide modular components 180 with electrical power and operably interconnect the various modular components 180 with the remaining elements of breathing device 160. Modular components 180 may comprise any function or combination of functions desired to treat a subject (e.g., a tracheotomy subject). For example, modular components 180 comprise at least one function selected from the group consisting of oxygen, humidification, nebulization, a battery, heat, ventilation, positive air pressure, wireless networking, vacuum pressure, suctioning, waste storage, cough assistance, enteral feeding pump, oral care, computer hardware and software, and biometric sensing. For example, the components 180 may comprise one or more of the positive pressure pump 120, the humidification and/or nebulization unit 122, the suction pump 124, the cough assist unit 126, the enteral feeding unit 128, and one or more oral care unit 130. Thus, a user may customize the function and performance of portable breathing device 160 by selectively coupling desired modular components 180 to portable housing 170.

In some embodiments, portable breathing device 160 comprises a display screen 174 that displays information to the user regarding the status of the various modular components 180. Display screen 174 may comprise a touchscreen whereby the user may manually adjust the settings of the various modular components 180. Portable breathing device 160 and/or modular components 180 may comprise manual gauges and controls, whereby a user may manually adjust the settings of breathing device 160.

In some embodiments, portable breathing device 160 comprises one or more biometric sensors that are configured to detect various biometric parameters of the subject and/or the modular components 180. For example, breathing device 160 may include one or more sensors (e.g., sensors 610 (FIG. 6)) that detect one or more of the oxygen levels of the subject, air temperature, humidity, air pressure, and sounds produced by the subject.

In some embodiments, the breathing device 160 comprises a single biometric sensor that is configured to detect a plurality of biometric parameters. In other embodiments, breathing device 160 comprises a plurality of biometric sensors, wherein each biometric sensor is configured to detect one or more biometric parameters.

Biometric sensors of the present disclosure may be positioned at various locations within breathing device 160 and/or on the subject as may be desired to receive accurate biometric data. For example, one or more biometric sensors are positioned within tubing 100. One or more biometric sensors may be positioned on tracheostomy tube 62, such that the sensor is positioned within the airway of the subject (e.g., sensors 610 as shown in FIG. 6). In some embodiments, biometric sensors may be attached directly to the subject, such as an oxygen sensor. In some embodiments, at least some of the biometric sensors may be positioned within the various modular components 180.

In some embodiments, portable breathing device 160 comprises circuitry and computer software whereby data from the various biometric sensors and modular components 180 are interconnected and accessible to the user via display screen 174. Accordingly, the user may access the data and make adjustments to the various modular components 180 as desired. In some embodiments, a user may access the data and make adjustments to the various modular components 180 using a remote computer device and a wired or wireless network connection.

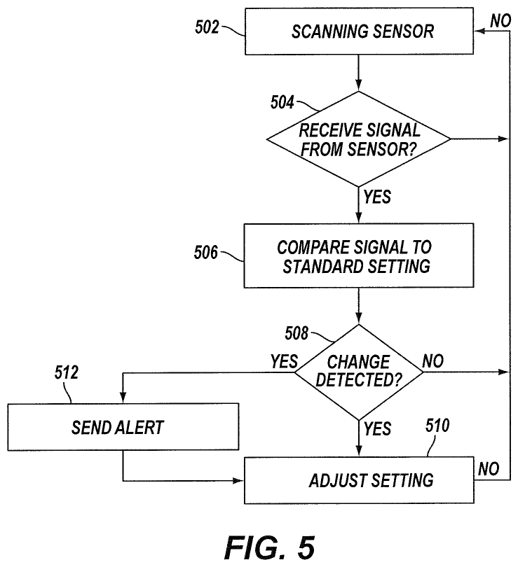

In some embodiments, portable breathing device 160 comprises a computer software program that is configured to perform a series of acts whereby a user may detect biometric parameters and adjust setting of the various modular components 180. Referring now to FIG. 5, a computer software method is shown. In some embodiments, the present disclosure comprises a computer-executable program having computer-executable instructions for scanning the one or more biometric sensors of breathing device 160 (at act 502). When a signal is detected (at act 504), the computer-executable program compares the value of the signal to the value of a standard setting. If a change in the value is detected (at act 508), the computer-executable program adjusts a setting of breathing device 160 to compensate for the change. The computer-executable program then continues scanning the one or more biometric sensors to detect additional changes in the value. When the value detected by the sensor is equal to the value of a standard setting, the computer-executable program goes into a standby mode and continues scanning the one or more biometric sensors.

In some embodiments, the computer-executable program comprises an act whereby an alert is generated in response to the detection of a value that is different than the value of a standard setting (at act 512). For example, the computer-executable program sounds an audible alert. In other embodiments the computer-executable program sends an alert to the display screen 174 of breathing device 160 (FIG. 4).

In some embodiments, breathing device 60, 160 (FIGS. 3, 3A, and 4) is operably coupled to a computer, cellular, or wireless network, whereby the computer-executable program generates and sends an alert to a remote computer device, such as a desktop computer, a nurse's station, a cellular phone, a tablet computer, or a smart device (e.g., a smart phone or smart watch). For example, breathing device 60, 160 comprises a transmitter 600 that is configured to attach to a portion of breathing device 60, 160, and is operably coupled to one or more biometric sensors 610, as shown in FIG. 6. In some embodiments, tracheostomy tube 62 comprises a biometric sensor 610 that is positioned within the airway 620 of the subject. Tracheostomy tube 62 comprises an electrical lead 630 that is embedded within a sidewall of tracheostomy tube 62 and operably connects biometric sensor 610 to an electrical contact 612 that is positioned external to the subject's airway 620. In some embodiments, the biometric sensor 610 and the electrical contact 612 may be directly electronically coupled to the breathing device 60, 160. In other embodiments, a transmitter 600 may be configured to clamp around tracheostomy tube 62 at the location of electrical contact 612. In some embodiments, transmitter 600 comprises a battery that provides electrical power to both transmitter 600 and biometric sensor 610. Signals from biometric sensor 610 are sent to transmitter 600 via electrical lead 630. In some embodiments, transmitter 600 comprises a wireless transmitter 602, whereby signals received from biometric sensor 610 are wirelessly transmitted to a remote computer device via transmitter 600.

The configurations and positions of biometric sensor or sensors 610, transmitter 600, and electrical lead 630 may vary depending upon the structure and configuration of breathing device 60, 160. Further, some embodiments of the disclosure provide an electrical receptacle in place of wireless transmitter 602, whereby a user may access biometric sensor 610 via the electrical receptacle. For example, a user may couple a separate wireless transmitter to transmitter 600 via an electrical lead attached to the electrical receptacle. In some embodiments, breathing device 60, 160 may include a plurality of transmitters positioned at various locations on breathing device 60, 160.

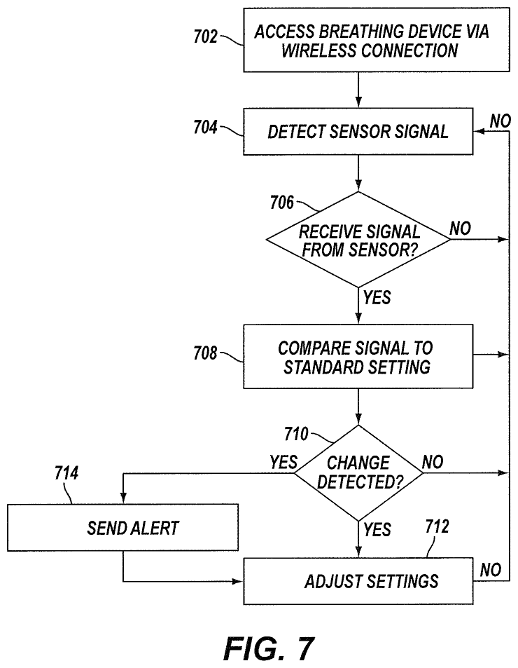

Some implementations of the present disclosure comprise a computer-executable program that is configured to perform a series of acts whereby a user may access and adjust settings on breathing device 60, 160 via a wireless computer device. Referring now to FIG. 7, a computer software method is shown. In some embodiments, the present disclosure comprises a computer-executable program having computer-executable instructions for accessing breathing device 60, 160 via a wireless connection (at act 702). The computer-executable program the scans the one or more biometric sensors of breathing device 60, 160 (at act 704). When a signal is detected (at act 706), the computer-executable program compares the value of the signal to the value of a standard setting (at act 708). If a change in the value is detected (at act 710), the computer-executable program adjusts a setting of breathing device 60, 160 to compensate for the change. The computer executable program then continues scanning the one or more biometric sensors to detect additional changes in the value. When the value detected by the sensor is equal to the value of the standard setting, the computer-executable program goes into a standby mode and continues scanning the one or biometric sensors.

In some embodiments, the computer-executable program comprises an act whereby an alert is generated in response to the detection of a value that is different than the value of a standard setting (at act 714). For example, the computer-executable program sends a wireless alert to a remote computer device. In some embodiments, the wireless alert is a text message. In other embodiments, the wireless alert is an email communication. Further, in some embodiments, the wireless alert is an audible alarm. The user may then access breathing device 60, 160 via the remote computer device to make adjustments to the settings of the components of the breathing device 60, 160 (e.g., components 180), as may be desired. The computer-executable program receives the settings from the remote computer device and adjusts the settings of the breathing device breathing device 60, 160 in accordance with the instructions received from the remote computer.

As discussed above, one or more of control of the breathing device 60, 160 and alerts from the breathing device 60, 160 may be sent and/or received from various electronic or computer devices (e.g., computer device 36 (FIG. 1)), such as, for example, smart phones, tablets, pads, computers, smart watches, and other electronic devices that may be controlled by a caregiver or the subject. In some embodiments, audio recordings of the subject (e.g., recorded with a microphone in the tracheostomy tube 62) may be sent to the various electronic or computer devices. In some embodiments, the computer device 36 may comprise a portable smart device (e.g., a smart phone, tablet, or watch) that may receive biometric data from the breathing device 60, 160. The smart device may be paired to the breathing device 60, 160 (e.g., via an application or “app” on the smart device) to receive and collect any information or data from the breathing device 60, 160 regarding the subject remotely from the breathing device 60, 160 (e.g., wirelessly). For example, alarms generated by the breathing device 60, 160 may be received by the smart device. In some embodiments, the smart device may be utilized to send commands to the breathing device 60, 160 and adjusts one or more settings of the breathing device 60, 160 remotely. In some embodiments, an application running on the smart device, which pairs the smart device to the breathing device 60, 160, may include an emergency help option that may be used to alert the proper emergencies authorities such that they can response to the subject. In some embodiments, such an emergency help option may be included on an input device of the control system 61 of the breathing device 60, 160. For example, the breathing device 60, 160 may enable the subject to produce a select audio sound that is a microphone sensor of the control system 61 of the breathing device 60, 160 where the control system 61 will alert the proper emergencies authorities in response to the selected audio sound (e.g., with a pre-taped audio recording indicating the location of the subject).

As mentioned previously, in some embodiments, a breathing device is provided having a one-way valve configured to prevent the subject from rebreathing exhaled gases. For some subjects, the physical structure of the subject's airway, or the structure of the tracheostomy tube prevents exhaled gases from being expelled via the subject's mouth. Accordingly, inhaled and exhaled air must travel through the tracheostomy tube. In some embodiments, tubing 100 comprises two or more lumens, wherein at least one lumen is configured to deliver fresh air to the subject, and wherein at least one other lumen is configured to remove exhaled air from the subject. The fresh and exhaled air is routed to their respective lumens via a one-way valve. In other embodiments, tubing 100 comprises a single lumen for delivering fresh air to the subject. For these embodiments, breathing device 160 comprises a one-way valve that is configured to vent exhaled gases into the environment.

Referring now to FIGS. 8A and 8B, a representative embodiment of a one-way valve 800 is shown. In some embodiment, one-way valve 800 comprises a housing 802 having a proximal end configured to receive or otherwise couple to tubing 100, and a distal end configured to receive or otherwise couple to tracheostomy tube 62. Housing 802 comprises a central chamber 804 that is in communication with the proximal and distal openings, and provides an air pathway through housing 802. Housing 802 comprises a floating valve 806 that is movable between an opened and a closed position. When in the open position (see FIG. 8A), floating valve 806 is slid distally within central chamber 804 thereby simultaneously unobstructing an ingress air pathway 810, and obstructing an egress air pathway 812. Air pressure 102 from tubing 100 pushes floating valve 806 distally. The distal movement of floating valve 806 is arrested by a distal stop 814. A distal end of ingress air pathway 810 comprises a flap valve 816 that is biased into an open position by air pressure 102. Air flows through ingress air pathway 810 and into the subject's airway 620 via tracheostomy tube 62.

When the subject exhales, air pressure 622 from the exhaled gases pushes floating valve 806 proximally to a closed position thereby simultaneously obstructing ingress air pathway 810 and unobstructing egress air pathway 812 (see FIG. 8B). Air pressure 622 further closes flap valve 816, thereby preventing exhaled gases from entering ingress air pathway 810. The proximal movement of floating valve 806 is arrested by a proximal stop. Expired air flows through central chamber 804 and out of housing 802 via egress air pathway 812. This process is repeated with each subsequent inhalation and exhalation of the subject.

In some embodiments, one-way valve 800 is positioned between the manifold 80 or housing unit 70 (FIG. 3) of the breathing device 60, 160 (FIGS. 3, 3A, 4) and the Y-port suction adapter. For example, a suction cannula may be fed through the tracheostomy tube without passing through one-way valve 800. Further, in some embodiments, one-way valve 800 is configured to direct exhaled gases into a separate lumen of tubing 100, as mentioned previously.

FIG. 9 is a schematic view of a breathing device 900 coupled to an airway access device or element 901 (e.g., a tracheostomy tube, a mask, an inhalation tent, an air hood, a nasal cannula, a tracheal tube, or other type of breathing tube) in communication with the airway of a subject. In some embodiments, the breathing device 900 and the airway access element 901 (e.g., tracheostomy tube 918) may be similar to the breathing devices 60, 160 and the tracheostomy tube 62 discuss above with references to FIGS. 3, 4, 6, 8A, and 8B. As depicted, the breathing device 900 includes a control unit 902 that may house and control the various components of the breathing device 900. For example, the control unit 902 maybe include one or more of the above-described ventilation unit, humidification unit (e.g., including a heating feature), nebulizer unit, suction unit, cough assist unit, pulse oximeter unit, oxygen concentrator unit, enteral feeding unit, and oral care unit (e.g., the positive pressure pump 120, the humidification and/or nebulization unit 122, the suction pump 124, the cough assist unit 126, the enteral feeding unit 128, and the one or more oral care unit 130 discussed above with reference to FIG. 3A).

Each unit housed by the control unit 902 may be operably connected to a manifold 906 (e.g., via tubing 904) that may at least one of combine, control, support, and organize the various tubing and/or wiring extending from the units housed by the control unit 902 to the subject (e.g., to the tracheostomy tube 918). For example, the manifold 906 may act to selectively power and/or selectively place the units housed by the control unit 902 in communication with one or more tubes (e.g., primary tube 910, secondary tube 912, or combinations thereof) connecting the tracheostomy tube 918 to the manifold 906. In some embodiments, the manifold 906 may be separate from the control unit 902. For example, the manifold 906 may be mounted on a swivel arm 908 such that the manifold 906 can move (e.g., translate and/or rotate) relative to the control unit 902. Such a configuration may enable to the manifold 906 and associated wiring and/or tubing to comply with movement of the subject in order to reduce the occurrence of tangling the tubing and/or wiring of the breathing device 80

The breathing device 900 may include one or more tubes (e.g., primary tube 910 and one or more secondary tubes 912) operably connecting the control unit 902 to the subject and/or the subject's airway (e.g., placing the one or more tubes in communication with the subject's airway). In some embodiments, the primary tube 910 may act to provide one or more treatments to the subject. For example, the primary tube 910 may be coupled with one or more of a ventilation unit, a humidification and/or heating unit, a nebulizer unit, and a suction unit. The secondary tube 912 may also act to provide one or more treatments to the subject. For example, the secondary tube 912 may be coupled with a cough assist unit.

The breathing device 900 may include a distal port 914 at which tubes 910, 912 connect with the tracheostomy tube 918. In some embodiments, a medication reservoir 918 may be connected to the breathing device 900 proximate the tracheostomy tube 918 (e.g., at distal port 914). For example, the medication reservoir 918 may be utilized in conjunction with the nebulizer unit to provide a dose of the medication into the subject's airway.

In some embodiments, the breathing device 900 may include an inline humidification and/or heating unit 916. The humidification and/or heating unit 916 may act to at least one of add moisture and heat a fluid (e.g., gas, air) as the fluid passes through the humidification and/or heating unit 916. In some embodiments, the humidification and/or heating unit 916 may be a removable and replaceable unit (e.g., a single use unit) the can be connected and disconnected from the breathing device 900. When implemented for humidification, the humidification and/or heating unit 916 may be initially provided with presoaked humidifying element, thereby eliminating the need to soak a humidifying element of the unit 916 prior to use as is generally required in convention humidification elements. In other embodiments, the breathing device 900 may include a fluid line to provide fluid to the humidification and/or heating unit 916.

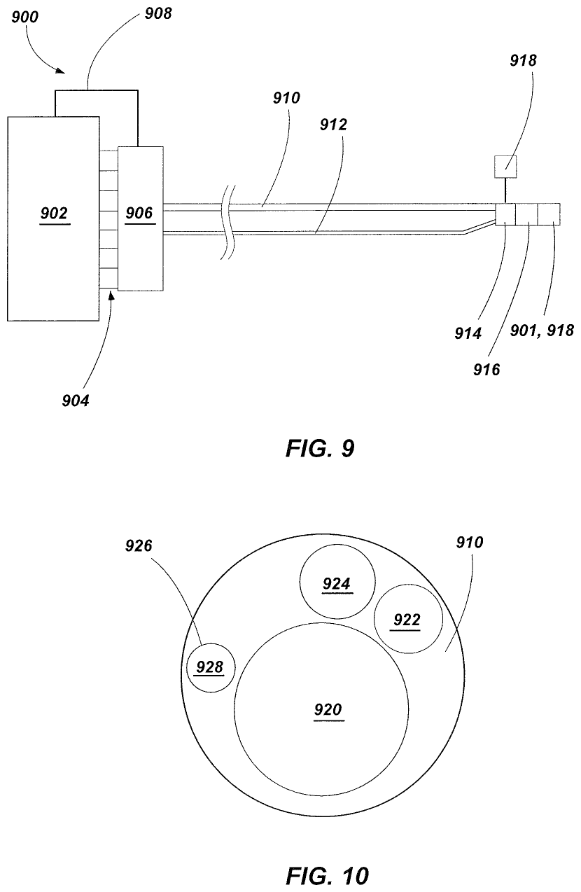

FIG. 10 is a cross-sectional view of a portion of the breathing device 900. In some embodiments, one or more tubes operably connecting the control unit 902 to the subject and/or the subject's airway (e.g., primary tube 910) may include multiple lumens for placing the control unit 902 into communication with the subject's airway. In some embodiments, one or more of the multiple lumens may be utilized for communication between various components of the breathing device 900 (e.g., for powering various portions of the breathing device 900). In some embodiments, primary tube 910 may include one or more of a first lumen 920 connecting the ventilation unit (and humidification unit, in some embodiments) to the tracheostomy tube 918, a second lumen 922 connecting the suction unit to the tracheostomy tube 918, and a third lumen 924 connecting the nebulization unit to the tracheostomy tube 918. The primary tube 910 may include a fourth lumen 926 housing a heating element (e.g., heating wire 928) for heating a fluid (e.g., air) as is it passes through one or more of the other lumens 920, 922, 924.

Embodiments of the present disclosure may be particularly useful in providing a breathing device incorporating multiple treatment components or units (e.g., a ventilation unit, a humidification unit (e.g., including a heating feature), a nebulizer unit, a suction unit, a cough assist unit, an enteral feeding unit, and an oral care unit) in a single breathing device. Such a breathing device may offer greater flexibility and mobility in the care of a subject by offering an integrated, portable, modular, mobile, ambulatory, lightweight, compartmentalized, and/or compact medical device as compared to other conventional breathing device that are generally only provided in separate, single units. When a subject requires multiple treatments, such conventional single units may be bulky and difficult, if not impossible, to transport with the subject, thereby, in some instances, confining the subject to one location where the devices are located.

Furthermore, such an integrated breathing device enables the monitoring and logging (e.g., for evaluation of compliance or noncompliance with a treatment schedule) of multiple treatment devices in a single unit that may provide alerts, notifications, and responses to the needs of the subject. For example, such an integrated breathing device may include one or more sensors for monitoring various parameters of the subject, such as oxygen blood saturation levels, air pressure, air temperature, air humidity, volume of air displaced, rate of flow, pH level, and audio monitoring of noises originating from the subject. In response to such parameters, the breathing device may automatically adjust the treatments devices, may start a new treatment, may discontinue a treatment, and/or may send an alert or notification to a caregiver or other individual monitoring the status of the subject. Further, a user and/or the subject may directly or remotely control the various components or units of the breathing device and may monitor the status of the subject and/or the breathing device from various computerized or smart devices.

While particular embodiments of the disclosure have been shown and described, numerous variations and alternate embodiments encompassed by the present disclosure will occur to those skilled in the art. For example, one having skill in the art will appreciate that the systems and methods of the present disclosure may be adapted for use with a mask, an inhalation tent, an air hood, a nasal cannula, a tracheal tube, or other type of breathing tube in place of a tracheostomy tube. The described embodiments are to be considered in all respects only as illustrative, and not restrictive. Accordingly, the disclosure is only limited in scope by the appended claims and their legal equivalents.

1-20. (canceled)

21. A breathing device, comprising:
a ventilator;
a cough assist unit;
a pump configured to apply a positive air pressure to operate the ventilator, and a positive air pressure followed by a negative air pressure to operate the cough assist unit; and
a control system for selectively operating the ventilator and the cough assist unit.

22. The device of claim 21, comprising a single housing in which the ventilator, the cough assist unit, and the pump is housed.

23. The device of claim 21, comprising a manifold operably connected to an output of the ventilator and an output of the cough assist unit.

24. The device of claim 23, comprising an airway device having a first end coupled to the manifold and a second end configured to couple to an airway of a patient.

25. The device of claim 24, wherein the airway device comprises a valve.

26. The device of claim 25, wherein the valve is a one-way valve.

27. The device of claim 24, comprising an oxygen component operably coupled to airway device via the manifold.

28. The device of claim 27, wherein the oxygen component comprises an oxygen concentrator.

29. The device of claim 24, comprising an enteral feeding unit configured to operably couple to a feeding tube of the patient.

30. The device of claim 24, comprising a sensor to monitor a blood saturation level of the patient.

31. The device of claim 30, wherein the sensor is operably coupled to the airway device.

32. The device of claim 30, wherein the control system comprises an alarm configured to sound an alert in response to data received from the sensor.

33. The device of claim 24, comprising a humidification unit operably coupled to the airway device via the manifold.

34. The device of claim 24, comprising a heating element operably coupled to the airway device via the manifold and configured to increase a temperature of a gas within the airway device.

35. The device of claim 34, comprising a humidification unit operably coupled to the airway device via the manifold and comprising the heating element.

36. The device of claim 24, comprising a suction unit operably coupled to the airway device via the manifold.

37. The device of claim 24, comprising a nebulizer unit operably coupled to the airway device via the manifold.
